(19) United States
(12) Patent Application Publication
(10) Pub. No.: US 2024/0290503 A1
(43) Pub. Date: Aug. 29, 2024

(54) ROBOTIC MEDICAL SYSTEM HAVING HUMAN COLLABORATIVE MODES

(71) Applicant: Leila Mae WEIN, Hedehusene (DK)

(72) Inventor: Leila Mae WEIN, Hedehusene (DK)

(21) Appl. No.: 18/656,527

(22) Filed: May 6, 2024

Related U.S. Application Data
(63) Continuation of application No. 17/311,201, filed on Jun. 4, 2021, now abandoned.

(30) Foreign Application Priority Data
Dec. 4, 2018 (IL) 263465

(51) Int. Cl. G16H 80/00; G16H 10/20; G16H 10/60; G16H 40/63; G16H 50/20

(52) U.S. Cl. CPC G16H 80/00; G16H 10/20; G16H 10/60; G16H 40/63; G16H 50/20

(57) ABSTRACT

A system for use in health care, configured to diagnose, instruct, and plan treatment for a patient. The system comprises a controller having three modes of operation: an autonomous decision-making mode; a patient interactive mode; and a doctor interactive mode. The controller is configured to use artificial intelligence in operation of the modes to analyze data obtained from at least some of the patient, health care provider, a diagnostic test, or a medical database. If indicated by an output from the analyzed data, the controller recommends diagnostic tests or clinical procedures, or provides instruction to robotically perform the test. Iterative operation of the system refines the output of analyzed data, enabling the system to provide a diagnosis and treatment plan for a patient. The system may be configured to process an outpatient visit to the emergency room from presentation to discharge.

### RELATED APPLICATIONS

This application is a continuation of U.S. Ser. No. 17/311,201, filed Jun. 4, 2021, which is the national phase of PCT/IL2019/051329, filed Dec. 4, 2019, which claims priority from IL 263465, filed Dec. 4, 2018. The contents of the aforementioned applications are incorporated herein by reference.

### FIELD OF THE INVENTION

The present invention relates to the field of medical robotics, especially to control systems that employ artificial intelligence in a health care setting to provide patient diagnosis, treatment plans, and instructions.

### BACKGROUND

Emergency room (ER) crowding has become a widespread problem in hospitals worldwide. Lack of facilities, delays, and diversions have increased to epidemic proportions. The problem is so detrimental that the lack of ER crowding is considered a measure of the success of a hospital or system. In the United States healthcare system, ER visits account for 11% of outpatient encounters, 28% of acute care visits, and 50% of hospital admissions. It is not uncommon for patients to be “boarded” in the ER for 48 hours or more until an inpatient bed becomes available. Many critical urban centers have no excess capacity to manage a natural disaster or terror event. A key factor in ER overcrowding is insufficient staff relative to the number of patients. Furthermore, in such a pressured environment, errors in diagnoses or inaccurate diagnoses are common.

Robotic solutions have been proposed to combat these challenges. Such solutions use robots to alleviate some duties of the nurses, doctors, and other staff, therefore attempting to increase efficiency and patient throughput in a medical setting.

For example, US 2011/0288417 discloses a robot system for evaluating patient status in an emergency room setting. The robot includes a monitor and an infrared camera that are coupled to a mobile platform. The robot also includes a controller that is programmed to autonomously move the mobile platform from one patient to another patient and process images captured by the infrared camera to determine if one or more patients needs assistance. By way of example, the robot can determine whether a patient is out of a bed, or in a position where he/she may fall out of the bed. The robot may be coupled to a remote station that allows an operator to move the robot and conduct a videoconference with the patient. The image captured by the infrared robot camera can be utilized to analyze blood flow of the patient. The robot can also be used to perform neurological analysis. The robot may operate in an exclusive mode, in which only one user has access control of the robot, or a sharing mode in which two or more users may share access with the robot. However, the robotic system is susceptible to human error and does not have any ability to improve decision-making capabilities over time. There is therefore a need for a comprehensive robotic system for use in an emergency room setting that overcomes at least these challenges.

US 2017/0323064 by Bates, for “Systems and methods for automated medical diagnostics”, describes, inter alia, systems and methods for providing patients with diagnostic measurement tools and audio/video guidance to perform clinical grade diagnostic measurements of key vital signs. These measurements are accomplished by using an automated remote end-to-end medical diagnostic system that monitors equipment usage for accuracy. The diagnostic system analyzes patient responses, measurement data, and patient-related information to generate diagnostic and/or treatment information that may be shared with healthcare professionals and specialists.

US 2014//0122109 by Ghanbari et al., for “Clinical diagnosis objects interaction”, describes, inter alia, a method for diagnosing a patient using a computer system. The method includes receiving symptoms and symptom history information from the patient; identifying a first set of diagnoses using the information; presenting the first set of diagnoses and a first set of questions to the patient in accordance with the information; receiving a first set of answers to the first set of questions; and identifying a second set of diagnoses and a second set of questions in accordance with the first set of answers.

US 2018/182475 by Cossler et al., for “Artificial intelligence based facilitation of healthcare delivery”, describes, inter alia, techniques employing artificial intelligence (AI) to facilitate reducing adverse outcomes associated with healthcare delivery. A computer implemented method comprises monitoring live feedback received over a course of care of a patient, wherein the live feedback comprises physiological information regarding a physiological state of the patient. The method further comprises employing AI to identify, an event or condition associated with the course of care of the patient that warrants clinical attention or a clinical response. The method further comprises generating a response, based on the identification of the event or condition, that facilitates reducing an adverse outcome of the course of care, and providing the response to a device associated with an entity involved with treating the patient.

Studies and technologies exist that explore human-robot interaction. For example, the article entitled “Human-Robot Collaboration: From Psychology to Social Robotics” by Judith Butepage et al, published in arXiv: 1705.10146, May 2017, describes an embodied approach to human-robot collaboration (HRC) that is inspired by psychological studies of human-human interaction (HHI). This reference categorizes interaction between robots and humans as either instruction, cooperation, or collaboration.

However, as of yet, an advantageous balance of use of these types of interaction for an emergency room setting and a strategy for implementing such has not yet been found. In particular, most medical technologies use a master-slave system, such as those used in minimally invasive surgery. There is also a psychological aspect in that humans must overcome a fear of robots making medical decisions. There is a need for a comprehensive robotic system for use in an emergency room setting that strikes a balance between human decision-making and robotic autonomous decision-making to achieve a system that is both more efficient and more accurate than a purely human system or purely robotic system would have been capable of.

The disclosures of each of the publications mentioned in this section and in other sections of the specification, are hereby incorporated by reference, each in its entirety.

### SUMMARY

The current system is configured for use in a medical care setting, and may cooperate with robotic control to enable diagnosis of a patient and to provide instruction and in some cases a treatment plan. The system generally comprises a processor, a memory storage, a user interface, and a controller having three modes of operation: an autonomous decision-making mode; a patient interactive mode; and a doctor interactive mode. The controller is configured to use artificial intelligence in operation of the modes to analyze data obtained from multiple sources, including the patient, health care provider, a diagnostic test, and a medical database. The controller uses the analyzed data to select a diagnostic test or clinical recommendation to be performed by a health care provider or a medical robot; and if indicated by an output from the analyzed data, the controller can instruct a medical robot to perform a diagnostic test. Iterative operation of the system refines the output of analyzed data, enabling the system to provide a diagnosis and treatment plan and/or instructions for a patient. The system may be configured to process an outpatient visit to the emergency room from presentation to discharge. Using iterative operation of the system, which comprises at least one alternation between at least two of the modes, the system is configured to achieve a diagnosis of the patient and at least one of a treatment plan or patient instructions.

It may be seen as an object of the present invention to provide a comprehensive robotic control system that is capable of both autonomous decision-making and collaborative decision-making with human(s), that is highly accurate and efficient, and improves its decision-making abilities over time. The system has the potential, for example, to drastically reduce overcrowding in emergency rooms and to increase diagnostic accuracy. With continued use over time and the application of machine learning and artificial intelligence, the system is capable of improving the rate and incidence of correct diagnosis and decreasing the incidence of incorrect diagnoses, prescription errors, such as prescribing patients medicines to which they are allergic, and reducing the rate of other common errors in treating patients under the stressful atmosphere of the emergency department. Whereas an exemplary embodiment uses the emergency room as a model setting, the system may be equally well applied to a number of health care environments. The system may be used at least for primary evaluation of the patient, for patient monitoring, and/or for validation and quality assurance of decision-making by human care providers.

The above described object and several other objects are intended to be obtained in a first implementation of the invention, by providing a control system for an emergency room setting having three different modes for decision-making. The three modes are: a fully autonomous mode, a patient collaborative interactive, information-exchange mode, and a doctor/health care provider collaborative, interactive, information-exchange mode.

The patient collaborative mode is an interactive mode for receiving information from the patient. The patient collaborative mode may be the initial default mode for receiving the patient into the emergency room. However, when it is clear that an urgent emergency is present, the fully autonomous mode may be used to first collect the basic essential information, using an attached medical robot to record such details as height, weight, vital signs such as blood pressure, temperature, heart rate and respiratory rate. By that means, the system can alert the hospital staff immediately, or sooner than would be achieved if the patient collaborative mode were entered first. Some questions are standard and will always be asked of all patients in the patient collaborative mode. Then, based on demographics and patient history, such as previous visits to the ER, hospitalizations, existing medical conditions, and vital signs, the controller may decide which questions to ask based on standard protocol, the patient's history, and on a comprehensive database of information relating to other patients.

The controller employs artificial intelligence or machine learning to learn over time, which questions are more useful to ask, based on which questions are more likely to lead to an accurate diagnosis, successful treatment plan, or useful knowledge extraction. The controller then stores the information received in a memory unit. In the patient collaborative mode, the controller may also address basic questions or concerns that the patient might have. For example, the patient may state his language of preference or ask to see a human staff member, or to alert a specific medical staff member from the same hospital who knows the patient's history, and the controller may respond accordingly. Big data compiled from other medical centers in a city, state, or country, and without associated identifying information, may be stored to use in real time and for future need. Such a feature may be especially useful, for instance, for identifying, tracking, and diagnosing patients during epidemics or toxic exposures, if many patients are present to the ER with similar symptoms in a short amount of time.

In addition to asking questions and processing the patient's answers, the system may be configured to answer questions posed by the patient regarding aspects of the patient's visit to the ER. In addition, the system may provide instructions to the patient. Such patient instructions may comprise a range of directions, for example, from simple guidelines on how to take a medication; warning signs to be aware of in case of symptom worsening; or interim inpatient directions or information on where and approximately how long he/she would need to wait for health care provider intervention.

If autonomous mode was not entered initially, after receiving patient information and potentially providing patient instructions in the patient collaborative mode, the system may utilize the fully autonomous mode, wherein the controller may make decisions regarding tests based on the current patient history, test results, and on an extensive information database with information relating to other patients. For example, in this mode the controller may decide which tests are needed, such as taking images with an x-ray, infrared or other imaging device, or performing an EKG. This mode may be used to take the patient through a plurality of tests that the controller may instruct a medical robot, or in certain circumstances, medical staff, to perform, including blood flow analysis, patient movement analysis, heart rate, and blood drawing. In the most ideal embodiment, this autonomous mode may be used for diagnosis of the patient, and in certain cases even for monitoring or supervising treatment, such that a human staff member would not need to be involved or be present at all. Such cases in which the fully autonomous mode would be especially useful for treatment include, for instance, administering IV fluids to a patient suffering from dehydration from a number of self-limited causes, prescribing antibiotics or other medicines for acute and non-life-threatening indications. The controller may employ artificial intelligence in autonomous mode to learn how to make better decisions or more accurate diagnoses using, for example, correlations and statistical analyses.

An important goal of the autonomous mode is not only to save medical staff time and make the ER run more efficiently, but also to standardize treatment and evaluation protocols so that potential diagnoses are not overlooked and essential treatments are not withheld, forgotten or neglected. An automated system as disclosed in the present application may also reduce problems caused by misreading of handwritten instructions or prescriptions. This is an example of how the system may be used for quality assurance and validation of health care provider decisions. The system may perform checks or review a diagnosis, test result interpretation, or treatment plans based on knowledge of past patient treatments, database mining, or scientific research article analysis. In autonomous mode, the controller may be adapted to triage and compare among all the patients waiting to be seen. The system may be adapted to assess the urgency of each patient's condition and the need to see a medical provider, and rank a particular patient's condition along with those of other patients waiting for treatment, such that the patient needing most urgent attention is given highest priority. Such priority is not limited to switching to doctor collaborative mode or human treatment of the patient, but may also include prioritization use of a robotic testing or imaging station if an insufficient number of stations is in operation relative to the number of patients present, prioritization of accessing a database, or any other time consuming task.

It is understood that at least the patient collaborative mode, and in some instances also the autonomous mode, may require at least minimal participation of the patient or, depending on the patient's condition, another human being who knows the patient's condition and can communicate with the controller, for example, by inputting data or responding to questions. The assisting person is preferably physically present with the patient, but may also communicate with the controller remotely, for example if the patient is unable to have an assisting person physically present and is instead on a video conference call with another individual such as a family member or friend. The system may also comprise a console or video screen for integrating the input from the third party directly to the system. The term “patient”, as used throughout this disclosure and as claimed, when relating to communication with the patient, is therefore meant to include a patient or any person acting on behalf of a patient, either physically present or remotely.

The doctor collaborative mode is an interactive mode that may enable the system to make intelligent decisions collaboratively with one or more doctors, nurses, or other medical staff members. There may be an interface on the controller for interacting with the physician or other medical staff, either by voice or by keyboard input, haptic input, etc. The controller may make suggestions based on the physician's or other health care provider's inputs, on the patient's history, and on its database comprising data on other patients. In the doctor collaborative mode there may be an exchange of information and shared representation between system and doctor. The level of interaction is based on a shared goal and may evolve into sharing interdependent subtasks and mutual learning between human and machine participants.

### Learning Paths, Switching Between Modes, and Sharing of Information Between Modes:

The learning path for the doctor collaborative mode and the autonomous mode are preferably different, and outputs of these different modes, questions output by the system or even suggested tests to perform may be contradictory, even for a patient with the same symptoms in the same situation. Just as a person works differently when working collaboratively compared with how he/she works alone, so does the controller. In the autonomous mode, the controller may strive to make the best decision based on the patient information, test results, the large medical database, and its statistical analysis capabilities. In the collaborative mode, the goal of the controller may be to most advantageously combine the expertise and knowledge of the doctor with the individual capabilities of the controller to arrive at the best decision. For example, this may include determining a percentage of representation of the controller and a percentage of representation of the medical provider when weighting judgments of the controller and medical provider, considering, for example, that a physician with 30 years of ER medicine experience would most likely arrive at better decisions, more accurate diagnoses, than an intern who is in his/her first year of medical practice. Such advantageous combination of the expertise of the doctor and the capabilities of the controller may also incorporate psychological statistics regarding human-machine interaction, personality profiles of doctors, level of comfort of a doctor with machine interaction, knowledge of limitations of doctors in general or of a particular doctor, knowledge of expertise or specialization of a doctor, and the like.

The different learning paths of the doctor collaborative mode and autonomous mode may be accomplished, for example, by using different training sets with a machine learning algorithm, setting different optimization goals, and/or using different optimization strategies. For example, if a goal of the doctor collaborative mode is for the system to arrive at an accurate diagnosis together with the doctor, and a sub-goal is to ask the doctor a question in a particular iteration that will be likely to lead to an accurate diagnosis, the training set for the particular machine learning algorithm employed may include a large number of conversations between doctors and the system, or if such data is unavailable, conversations between two doctors and which of these conversations led to accurate diagnoses. The machine learning or AI algorithms used in the doctor collaborative mode and in the autonomous mode may be the same or different, but if the same algorithm is used to accomplish the same task in two different modes then the algorithm will employ a different model for each mode using different parameters. If the learning paths in different modes are the same in a particular instance to solve a particular problem, this may be due to lack of sufficient training data, and can be resolved by the system collecting more data over time for future use in training sets.

The learning path for the patient collaborative mode is preferably different than that for the doctor collaborative and autonomous modes and outputs of these different modes, such as, for instance, questions output by the system or even suggested tests to perform, may be contradictory even for a patient with the same symptoms in the same situation, since the goal of the artificial intelligence aspect in the patient collaborative mode may be to ask useful questions or obtain medically relevant information from the patient that is likely to lead to an accurate diagnosis, successful treatment plan, and/or useful knowledge extraction. The system may need to take into account both the varying capacities and propensities for different patients to interact with a controller, especially a patient who is in pain, unable to move freely, unable to speak, unfamiliar with computer interfaces in general, or otherwise has difficulty communicating with the controller, especially since some patient responses may be oral and some provided via keyboard input. In such a situation, a human mediator, who is preferably physically present but may also be remote, may be employed to facilitate communication between the patient and the controller. In patient interactive mode, the controller may learn to ask questions that the patient is capable of answering. For example, most patients can indicate they are in pain, but not everyone can describe the quality of the pain, sharp, burning, dull, achy, or its intensity, on a scale of 1 to 10. Thus, if a patient cannot answer the questions output by the controller, the controller may learn to ask different subsequent questions to come to the best conclusions regarding the patient's condition. This is similar to the response of a health care provider with a patient intake evaluation.

The different learning paths of the patient collaborative mode and autonomous mode may be accomplished, for example, by using different training sets with a machine learning algorithm, setting different optimization goals, and/or using different optimization strategies. The machine learning or AI algorithms used in the patient collaborative mode and in the autonomous mode may be the same or different, but if the same algorithm is used to accomplish the same task in two different modes, then the algorithm will employ a different model for each mode using different parameters. If the learning paths in different modes follow the same pattern or sequence in a particular instance to solve a particular problem, such a limitation may be due to lack of sufficient training data, and can be resolved by the system collecting more data over time for use in future training sets.

When the controller then switches to health care provider interactive/collaborative/information-exchange mode, and the medical provider asks the controller questions regarding the patient's condition, the controller may be able to indicate that the patient was incapable of providing specific information, and that instead the controller was able to acquire other data. For example, if the patient proves incapable of answering, namely, by providing an answer of “I don't know” or by having no response, the controller may tailor the questions to assess the patient's mental status, asking for example, what is the date, who is the president, what is the patient's present location, and so forth. From these questions, the controller may determine the patient's alertness and identify possible mental status changes indicative of a specific type of disease process or clinical status.

This level of sophistication is acquired by the controller via machine learning and other forms of artificial intelligence. The term “machine learning” used throughout this description and as claimed may include any supervised or unsupervised machine learning algorithm, such as support vector machines, k-means clustering, logistic regression, linear regression, decision trees, naïve Bayes classifier, random forest, KNN, or the like. The term “artificial intelligence” used throughout this description and as claimed includes artificial neural networks, regression algorithms, instance-based algorithms, decision tree algorithms, clustering algorithms, association rule learning algorithms, ensemble algorithms, deep learning algorithms, and the like. Repetitive operation of the various modes, and repetitive operation of loops of two modes, results in refinement of the data collected as analyzed data or analyzed information. In this manner, raw data is converted to more useful analyzed data and controller outputs.

The patient's current vital signs may become more meaningful after they have been compared to the same parameters last week, a month ago, etc. If the patient's blood pressure or temperature is rising or dropping, the current values become more useful when presented as a continuum. Data collected from the patient and the trend data or other analyzed information is available to all three modes. Other examples of how the system of the present disclosure may make use of analyzed data is to look at clusters of abnormal lab or vital sign values and provide a list of potential diagnoses. In essence, the thought process that a health care provider would use to diagnose a patient may be used by the system in autonomous mode, for example via an artificial neural network, as it learns over time how to acquire and process clinical data. However, the diagnoses arrived at by the system in autonomous mode or in doctor collaborative mode are likely to be more accurate than from a doctor alone, since the system has access to larger amounts of data and can process that data extensively.

Further, based on the patient's answers in patient interactive mode, the controller may provide medical information to the patient, as well as to a medical provider, regarding the patient's condition, possible diagnoses, estimated time course of treatment, estimated wait time, or other relevant details. This aspect of the system is an important reassurance to someone with an acute medical issue, who has no knowledge of his/her condition and may well experience fear of the unknown. Being provided with information regarding potential causes, time course, or even simple procedural information in such a state, may lead to better outcomes by reducing stress levels.

In all scenarios, both collected patient information and analyzed data may be available to, and used by, all three modes, both in use with a patient and also as training data for training AI or machine learning algorithms. Furthermore, information input by a medical provider, judgments made by a medical provider, and judgments made by the system may be available to all three modes. There is no inherent limit in the system to the number of iterations or amount of shared information of the system between modes; the limit may therefore be determined by practicality factors such as computing power, computing speed, etc. The system should preferably strive to arrive at the simplest solution with the least number of iterations that is capable of providing an accurate result according to the criteria of Occam's razor. The system is designed to assess at the outset, or after minimal evaluation, how much time should be taken for detailed evaluation vs. providing urgent or emergent treatment recommendations.

A provisional diagnosis given by the doctor while in collaborative mode may be added to the information gathered by and available to the controller for use in autonomous mode. Patient data collected in patient collaborative mode may be accessible to the doctor in doctor collaborative mode. Test results obtained by the controller in autonomous mode may be accessible to the doctor in doctor collaborative mode. All of these pieces of information are analyzed, and the analyzed information may also accessible to any mode. If a doctor wishes, he may also access statistical analysis, graphs, or calculations that the controller has generated. With each successive patient evaluated and treated, the system learns and is able to apply the learning to new situations. The findings from each patient may be further added, anonymously and with the appropriate permissions, to the at least one medical database. However, just because data may be accessible by all three modes does not necessarily mean that it is used; this is determined according to the results of different learning paths used by the different modes.

The controller may “ask for help” and suggest a switch from autonomous mode to doctor collaborative mode if it realizes its own limitations, for example, if two possible responses have equal weight, but which are mutually contraindicative suggested treatments, and the controller is not sure which one to choose, or if the controller has never encountered such a set of symptoms and does not have a suggested course of action, or it realizes that the patient is in a dangerous situation requiring human assistance. The realization of its own limitations may be accomplished, for example, by algorithms being programmed to incorporate a risk assessment based on known limitations of the system, or based on lack of experience of the system for a particular task, or lack of data collected with regards to a particular task. The controller may provide many outputs. Some examples of outputs are: a percentage of confidence in an output, an estimated error of an algorithm, a probability of patient mortality based on collected data and/or test results, a degree of urgency for treatment, or a prioritization of the patient within a triage. It may also provide two equally likely diagnoses and the percentage confidence associated with each, or other such outputs that will help the doctor to understand why the controller is suggesting a switch to doctor collaborative mode. The doctor may either accept the switch to doctor collaborative mode, or the system may be programmed to refuse to continue in autonomous mode in certain scenarios that are deemed particularly dangerous based on behavioral or psychological statistics regarding human-machine interaction. Although it would be an unlikely scenario, the doctor may choose to override this suggestion and allow the controller to continue in fully autonomous mode.

A health care provider may intervene and request doctor collaborative mode at any time during use of the system. For example, if a patient presented with an unusual collection of symptoms in autonomous mode, the doctor may ask the controller to perform a specific additional search that the controller appeared to miss based on its programming or learning. Although it is not an ideal implementation of the system, the doctor may also have the capacity to, at any time during use of the system, leave the controller out of the decision-making process, exiting the collaborative mode at an appropriate point in the evaluation, and thereafter interacting with the patient directly.

Furthermore, the system may be configured to operate in multiple capacities, initially as an intake module, and subsequently in a mobile patient observation capacity. Collaboration with the patient in each scenario may require different processes, methods of interaction with the patient and/or doctor, and outputs. For example, in the intake mode, questions may be directed at gathering information and arriving at an initial course of action. In the observation scenario, the controller may be required to collect and analyze patient data and/or instruct a robot to perform regular tests, for example, to generate follow-up questions about changes in signs and symptoms over time, either as a result of treatment or while waiting for a medical provider to assess the patient. The system may also recommend tests that require an outside testing location or facility beyond the capabilities of an integrated or local robot. In the patient observation capacity, the controller may switch between all three modes to provide long-term monitoring and optimal interim care, for example, for a patient with a less serious condition awaiting attention by medical staff. The system may be configured to process an outpatient visit to the emergency room from presentation to discharge.

The controller may switch from fully autonomous mode to patient interactive mode whenever the system determines that additional information from the patient would be useful in determining the next step in the decision-making process. The controller may then switch back from patient interactive mode to autonomous mode, when it determines that it has sufficient patient information for the next step in the decision-making process. The patient may also be allowed to input requests to the controller during autonomous mode, for instance, if he feels he needs help urgently, or if he prefers blood to be drawn from the other arm, or any similar such request. If the controller determines that during patient interactive mode, the patient stops responding or otherwise refuses to cooperate, it may switch to autonomous mode or doctor collaborative mode, and/or alert a medical provider of the problem.

These three modes—doctor collaborative mode, patient collaborative mode, and autonomous mode—may operate interchangeably in the various capacities of the control system, e.g., first as an intake module and later on in a mobile patient observation capacity. The controller may switch from autonomous mode to doctor-interactive mode based on interactions with a patient under observation. In some emergency departments, more than one controller may be employed at a given time, and activities may be coordinated among specific controllers. In such a scenario, each controller operating in autonomous mode may receive information regarding the status of each patient currently waiting for treatment and use this additional information in a triage capacity. Further, comparing patient data between and among controllers may allow faster identification of an epidemic crisis or other epidemiological event. Efficiency of ER function may be further enhanced when multiple controllers communicate with each other. Preferably, one or more controllers provide instructions to more than one medical robot adapted to perform specific task(s), such that if several patients need the same test or treatment able to be administered by a medical robot, the work could be queued among the medical robots in the most efficient manner.

Different controllers may have different levels of intelligence or decision-making capacity, depending on how long a particular controller has been locally learning its function at the station it is operating, how interactive the controllers are with each other, and how much the overall system is able to co-ordinate each controller's learning history with the other control stations. Thus, it is assumed that a controller that had been operative in the same department for many years would be ‘smarter’ and make better decisions than a newly inaugurated one. Obviously, the more interactive the control stations are with each other, the more accurate and useful the results would be by learning from each other.

An exemplary system comprises a hardware controller, configured to operate in at least two of the three modes described herein above, during the course of diagnosis and treatment of a single patient, and more advantageously with all three. The system is configured to use artificial intelligence or machine learning to alternate between and among the modes, recognizing its limitations and capabilities. In the two collaborative modes, bidirectional information exchange occurs between the controller and a human being, either the patient or the health care provider. For a basic example, in an exemplary implementation, if the system operating in patient-collaborative mode recognizes that it has reached the limit of information it can obtain from the patient, it will switch to autonomous mode. In autonomous mode, the system will process the information it has received from the patient, compare it with information accessed from at least one medical database, and decide the next step, which could be to instruct a medical robot, or in certain instances, a medical staff member, to carry out a specific diagnostic test. It is understood that the term ‘medical database’ is a general designation for virtual libraries of information on demographics, lab results, radiological images, micrographs of pathology specimens, surgical findings, and any other medically-relevant information collected, anonymously and with permission, from a group or many disparate groups, of healthy individuals and those with medical diagnoses.

During actual operation of the system in an exemplary implementation, interactions may take place between the system in patient-collaborative mode and the patient; between the system in doctor-collaborative mode and the medical staff; and between the system in autonomous mode and the robot performing tests which provide diagnostic results. Each of these interactions form loops of iterative processing, the output of which feed into the controller's memory and are then used as input in higher level processing of data. The higher level processing may involve the system alternating among modes, asking specific disease-related questions of either the doctor, the patient, or querying such information in the medical database. In the higher-level processing the system analyzes data and draws conclusions. The system may also perform online searches to access generally available medical information that would aid in diagnosis.

With each iteration of processing in various modes, the system arrives closer to an accurate diagnosis, or list of differential diagnoses. The system has inherent flexibility in that it provides the same accurate diagnosis, independent of the paths it follows to arrive at the diagnosis. Different paths or different combinations or orders of mode implementation may be used. Various external factors may affect the combination of modes used, or the order of the modes, such as doctor availability or patient ability to respond. In any case, each of the modes or combination of modes is optimized to provide the best diagnosis and treatment plan possible in that mode or combination of modes. Each mode is programmed to improve its efficiency and accuracy of operation using artificial intelligence, such as machine learning. As such, in doctor-collaborative mode for example, the system may come to recognize the psychological profile of specific health care providers. Thus, each individual system operating in a particular health care setting will develop a unique set of operating parameters based on the illnesses diagnosed and treated, the health care providers, the patient load, and other factors unique to its environment. Thus, a system at a tertiary care center may be more adept at diagnosing rare diseases than one in a community-based hospital.

Implementations of the system of the present disclosure are amenable and configurable to operate via tele-medicine, such that the autonomous mode and the doctor collaborative decision-making mode may be accessed remotely and used to consult with human health care providers or other systems in different locations. With time, accessing information via the at least one medical database is not limited to a static library or libraries of medical data, but is a dynamic collection of information that is constantly being updated. The knowledge gained by each system operating on its own may be fed into a central, electronic library of medical data, both general and specific. Thus, over time, a ‘new’ or ‘young’ system may be as diagnostically clever as an ‘old’ system that has been in operation for a period of time.

In some implementations, the controller may be further configured to do at least one of the following. In the autonomous decision-making mode, the controller may improve the decision-making capabilities of the robotic control system based on at least one of a) the data, b) statistical analysis performed by the robotic control system, and c) the diagnostic tests performed by the at least one medical robot. In the doctor collaborative mode, the controller should incorporate the expertise of the at least one health care provider with the data and with the statistical analysis of the robotic control system to improve collaborative decision-making capabilities. In the patient collaborative mode, the controller should output questions to the patient likely to lead to at least one of an accurate diagnosis, accurate treatment plan, and patient instructions. The learning paths for autonomous mode, doctor collaborative mode, and patient collaborative mode are generally different.

In some implementations, at least one of collected patient data, analyzed information, doctor judgments/decisions, and system judgments/decisions, should be available to doctor collaborative mode, patient collaborative mode, and autonomous mode for use in at least one of making further judgments/decisions, or/and as training set data for artificial intelligence algorithms. A judgment or decision is defined as an input to the system that is not raw data (i.e. has been arrived at by at least some analysis or calculation) and may be final, for example a patient diagnosis or treatment plan, or non-final, for example, an intermediary judgment.

In some implementations, due to different learning paths, the system's method of arriving at a diagnosis or treatment plan may differ for a second patient with the same symptoms in the same situation as a first patient. The methods comprise system outputs using various combinations of modes. For a first patient, the system may use a first mode or first sequential combination of modes, which may differ from a second mode or a second sequential combination of modes used for a second patient. For a given patient with given symptoms, the system may be configured to use a first mode or first sequential combination of modes, and a second mode or a second sequential combination of modes, to perform different methods that are driven by different optimization goals or optimization strategies yet arrive at the same ultimate treatment plan or diagnosis. The first mode or sequential combination of modes improves diagnosis over time using artificial intelligence according to a first optimization goal or strategy, and the second mode or sequential combination of modes improves methods over time using artificial intelligence according to a second optimization goal or strategy, such that diagnostic accuracy is improved.

The robotic control system may be configured to provide information to the patient in response to a patient query in at least one of the patient interactive mode and the autonomous mode. In autonomous mode, the system is adapted to exchange analyzed data with, and to learn from, any number of other robotic control systems. In some implementations, the controller is configured to be used in at least one of the modes for long-term patient monitoring in a health care setting. In the autonomous mode, the controller may be configured to instruct the medical robot to perform any number of diagnostic procedures, comprising at least one of blood drawing, imaging studies, and vital sign acquisition. The controller may also instruct at least one medical robot to perform any number of therapeutic procedures, comprising at least one of prescribing or dispensing medication; providing written or oral instructions; and administering IV fluids.

In the case that iterative sharing of the exchanged, analyzed data between the patient collaborative mode and the autonomous mode results in different output from iterative sharing of the exchanged, analyzed data between the doctor collaborative mode and the autonomous mode, comparison of output from the modes enables achievement of improved diagnostic accuracy. Iterative operation of the controller may incorporate artificial intelligence to reach at least one of the diagnosis and the treatment plan, or the patient instructions. The system may arrive at a single, definite diagnosis, or may provide a human medical provider with a narrowed list of differential diagnoses. The artificial intelligence may comprise machine learning. Based on iterative operation, the controller is configured to switch among the three modes. The decisions of whether to switch to a different mode, when to switch to a different mode, and which mode to switch to, are made by the system using at least collected patient data and artificial intelligence, such that diagnostic accuracy is improved.

The controller may be further configured to provide patient instructions and report recommendations to at least one health care provider, based on the analyzed data provided by at least one of the decision-making modes. The system may further comprise a user interface adapted to exchange the data between the controller and at least one of the patient and the health care provider. The data comprise any of the patient's current age, gender, height, weight, BMI, blood pressure, heart rate, blood laboratory test results, imaging study results, biopsy test results, a previous medical diagnosis, or the patient's past medical data.

Data analysis comprises at least one of computational statistics, data mining using exploratory data analysis, or data mining through unsupervised learning, the data being obtained from at least one of the patient, the health care provider, the patient's previous medical records, the at least one medical database, or previous learning by the robotic control system Alternation among the modes is determined by the system obtaining maximum data/information from a given mode, whereupon it switches to a different mode, the controller beginning its operation in the patient collaborative mode, switching to the autonomous mode, and, if indicated, switching to the doctor collaborative mode.

Further disclosed is a method for automated assessment of a patient, comprising the steps of: 1) providing a controller capable of iteratively alternating among three decision-making modes, the modes comprising: a patient information-exchange mode, an autonomous mode, and a health care provider information-exchange mode,
2) using the controller to obtain data from the patient and from one or more medical databases,
3) using artificial intelligence and statistical analysis capabilities of the controller, analyzing the data, at least one of the data or the analyzed data being exchanged among at least two of the modes;
4) using iterative operation of the controller to: a) in the autonomous decision-making mode, make decisions based on i) the analyzed information and ii) input from at least one of the collaborative modes; b) in the health care provider information-exchange mode, make decisions based on the analyzed information and incorporating the expertise of at least one health care provider; and c) in the patient information-exchange mode, output questions, analyze answers provided by the patient, and provide instructions to the patient;
5) performing at least one iterative operation in at least two of the modes; and
6) arriving at at least one of a diagnosis of the patient, a treatment plan, or patient instructions.

The method may further comprise three additional steps: 1) in the autonomous decision-making mode, improve decision-making capabilities of the robotic control system based on at least one of a) the data, b) statistical analysis performed by the robotic control system, and c) the diagnostic tests performed by the at least one medical robot; 2) in the doctor collaborative mode, incorporate the expertise of the at least one health care provider with the data and with the statistical analysis of the robotic control system to improve collaborative decision-making capabilities; and 3) in the patient collaborative mode, output questions to the patient likely to lead to at least one of an accurate diagnosis, accurate treatment plan, and patient instructions, wherein the learning paths for autonomous mode, doctor collaborative mode, and patient collaborative mode are different.

The method may further comprise the step of providing at least one of the collected patient data, analyzed information, and controller judgments/decisions, to doctor collaborative mode, patient collaborative mode, and autonomous mode for use in at least one of i) making further judgments/decisions and ii) as training set data for artificial intelligence algorithms. Due to different learning paths, the method of arriving at a diagnosis or treatment plan in a first mode or first sequential combination of modes, may be different for a second patient with the same symptoms in the same situation. The method may further comprise the following steps: 1) providing information to the patient in response to a patient query in at least one of the patient interactive mode and the autonomous mode, 2) the controller in autonomous mode exchanging analyzed data with, and learning from, any number of other controllers; and 3) instructing at least one medical robot to perform any number of diagnostic procedures, comprising at least one of blood drawing, imaging studies, and vital sign acquisition. If iterative exchange of the exchanged, analyzed data between the patient collaborative mode and the autonomous mode results in different output from iterative exchange of the exchanged, analyzed data between the doctor collaborative mode and the autonomous mode, comparison of output from the modes achieves improved diagnostic accuracy. The method may further comprise iterative operation of the controller using artificial intelligence, comprising machine learning, to reach the diagnosis and at least one of the treatment plan, or the patient instructions. The instructions to the patient comprise any of: to fill a prescription, to take a prescription, to return for a scheduled follow-up visit, to carry out routine home care tasks, to contact a health care provider, to make an appointment, to allow a medical robot to carry out a test such as blood drawing, to transfer to a different department, or other instructions that are routinely provided to a patient by a human provider in a medical setting.

The controller, based on the iterative operation, switches among the modes, and wherein the decision of at least one of i) whether to switch to a different mode, ii) when to switch to a different mode, and iii) which mode to switch to, is made by the controller using at least collected patient data and artificial intelligence, such that diagnostic accuracy is improved. The method may comprise any of the patient's current age, gender, height, weight, BMI, blood pressure, heart rate, blood laboratory test results, imaging study results, biopsy test results, a previous medical diagnosis, or the patient's past medical data. Analyzing of the data comprises at least one of computational statistics, data mining using exploratory data analysis, or data mining through unsupervised learning, the data being obtained from at least one of the patient, the health care provider, the patient's previous medical records, the at least one medical database, or previous learning by the controller Alternating among the modes is determined by the controller obtaining maximum data/information from a given mode, whereupon it switches to a different mode, the controller beginning its operation in the patient collaborative mode, switching to the autonomous mode, and, if indicated, switching to the doctor collaborative mode.

In some implementations, the system is configured for use in a medical care environment, the system comprising a controller, adapted to implement:
(a) a patient collaborative decision-making mode in which the controller exhibits bidirectional information exchange with a patient;
(b) an autonomous decision-making mode in which the controller is fully autonomous; and
(c) a doctor collaborative decision-making mode in which the controller exhibits bidirectional information exchange with a medical staff member;
   the controller being configured to obtain information from the patient and from at least one medical database, and to perform statistical analysis on the information, the information being made accessible to the patient collaborative decision-making mode, to the autonomous decision-making mode, and to the doctor collaborative decision-making mode; wherein the controller employs artificial intelligence to increase the likelihood of arriving at at least two of a more accurate diagnosis of the patient, an accurate treatment plan, or patient instructions, using output generated by: (i) the autonomous decision-making mode, providing output based on the information and statistical analysis capabilities of the controller; (ii) the doctor collaborative mode, incorporating the expertise of the medical staff member with the information and with the statistical analysis capabilities of the controller; and (iii) the patient collaborative mode, outputting questions, obtaining information, and answering questions.

In an exemplary embodiment, the system comprises a robotic system for use in an emergency room, comprising: a controller, adapted to implement: (a) a patient collaborative decision-making mode in which the controller exhibits bidirectional information exchange with a patient; (b) an autonomous decision-making mode in which the controller is fully autonomous; and (c) a doctor collaborative decision-making mode in which the controller exhibits bidirectional information exchange with a medical staff member. The controller is configured to obtain information from the patient and from at least one medical database, and to perform statistical analysis on the information, the information being made accessible to the patient collaborative decision-making mode, to the autonomous decision-making mode, and to the doctor collaborative decision-making mode. The controller employs artificial intelligence to increase the likelihood of arriving at at least two of a more accurate diagnosis of the patient, an accurate treatment plan, or patient instructions, using output generated by: (i) the autonomous decision-making mode, providing output based on the information and statistical analysis capabilities of the controller; (ii) the doctor collaborative mode, incorporating the expertise of the medical staff member with the information and with the statistical analysis capabilities of the controller; and (iii) the patient collaborative mode, outputting questions, obtaining information, and answering questions.

In this disclosure, a ‘medical care environment’ comprises any setting in which medical care is provided. Examples include emergency departments, inpatient hospitals, outpatient clinics, and urgent care clinics. Further relevant definitions for the purposes of the present disclosure: ‘medically relevant data’ or ‘data’ is defined as information used in a medical setting for the purposes of diagnosing or treating a patient. Such data may be a) routinely collected from a patient, friend or family member by asking questions; b) retrieved from a database of clinical information; c) findings in a medical or scientific research paper; or d) gathered from any typical source used in a medical or clinical environment. ‘Analyzed data’ comprises collected information from at least one source that has been processed by statistical analysis, comparisons, graphing or identification of trends over time. ‘Health care provider’, ‘doctor’, ‘medical staff member’, or similar term defines any human provider of care in clinical or outpatient environment. ‘Artificial intelligence’ is used by a controller or processer to make decisions based on or using at least one of machine learning, reactive machines, limited memory, theory of mind, and self-awareness. ‘Medical database’ is understood to be a collection of medically relevant information. ‘Differential diagnosis’ is understood to be the process of differentiating between two or more conditions which share similar signs or symptoms, or distinguishing of a particular disease or condition from others that present similar clinical features. In the present disclosure, ‘diagnosis’ is broadly understood to include a provisional diagnosis, a differential diagnosis, or a final diagnosis. ‘Provisional diagnosis’ is understood to be conditional for reasons such as that the system would need more information, or because the patient's clinical presentation at the time of evaluation is consistent with more than one condition. A “treatment plan” or “patient instructions” may be in accordance with any of a final diagnosis, differential diagnosis, or provisional diagnosis. Generally, a treatment plan is provided in accordance with a final diagnosis and patient instructions are provided in accordance with a provisional or differential diagnosis, but the scope of the present invention is not limited thereto. For example, a treatment plan in accordance with a provisional diagnosis or differential diagnosis may include scheduling the patient for a follow up appointment with a doctor. The system in this disclosure may access one or more such databases, examples of which are individual electronic hospital records; anonymous individual or pooled records maintained by HMOs, insurance companies, or other organizational health providers; and governmental databases such as pubmed.gov, Food and Drug Administration, or the Centers for Disease Control. ‘Clinical procedure’ or ‘diagnostic test’ comprise any number of routine diagnostic examinations. Examples include blood drawing for laboratory tests, X-ray, ultrasound, or urine collection.

In this disclosure, the term controller or computer may refer to, be part of, or include an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor (shared, dedicated, or group) that executes code; memory (shared, dedicated, or group) that stores code executed by a processor; other suitable hardware components, such as optical, magnetic, or solid state drives, that provide the described functionality; or a combination of some or all of the above, such as in a system-on-chip. The term code, as used above, may include software, firmware, and/or microcode, and may refer to programs, routines, functions, classes, and/or objects. The term shared processor encompasses a single processor that executes some or all code from multiple modules. The term group processor encompasses a processor that, in combination with additional processors, executes some or all code from one or more modules. The term shared memory encompasses a single memory that stores some or all code from multiple modules. The term group memory encompasses a memory that, in combination with additional memories, stores some or all code from one or more modules. The term memory may be a subset of the term computer-readable medium. The term computer-readable medium does not encompass transitory electrical and electromagnetic signals propagating through a medium, and may therefore be considered tangible and non-transitory. Non-limiting examples of a non-transitory tangible computer readable medium include nonvolatile memory, volatile memory, magnetic storage, and optical storage.

The apparatuses and methods described in this disclosure may be partially or fully implemented by one or more computer programs executed by one or more processors. The computer programs include processor-executable instructions that are stored on at least one non-transitory tangible computer readable medium. The computer programs may also include and/or rely on stored data.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure.

### BRIEF DESCRIPTION OF FIGURES

The present invention will be understood and appreciated more fully from the following detailed description, taken in conjunction with the drawings in which:

FIG. 1 is a diagram showing several possible paths and steps in human-controller interactions;

FIG. 2 is a schematic representation of the flow of information in an exemplary implementation of the system, showing multiple integrated loops of information exchange;

FIG. 3 is a schematic representation of the three modes of operation, showing possible ways in which the three modes of operation perform iterative loops of bidirectional information exchange;

FIG. 4 shows an exemplary flow chart of an exemplary method of the system; and

FIG. 5 illustrates a more detailed exemplary implementation of step 114 of FIG. 4, which is an exemplary flow chart of a method of the system, detailing the steps involved in carrying out specific tests.

### DETAILED DESCRIPTION

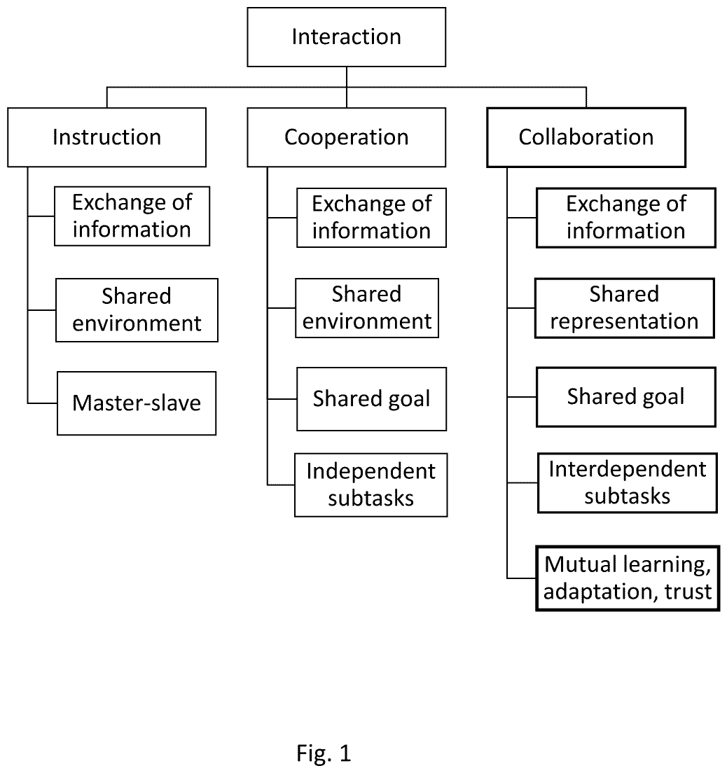

Reference is first made to FIG. 1, which shows a description of human-robot interactions, indicating several possible paths and levels of such a relationship. The two collaborative modes of the present disclosure use features of the collaboration column in FIG. 1. In a collaboration, the robotic system and human beings, in this case either the patient or the health care provider (doctor, nurse, or other medical professional), interact in a collaboration. Information is exchanged, such that each component of the interaction provides specific information. The robotic system and human beings accomplish tasks together toward a shared goal. The process entails mutual learning, adaptation, and eventually trust, as further outlined herewithin below. Thus, in this context, ‘collaboration’ has a distinct definition as will be clarified.

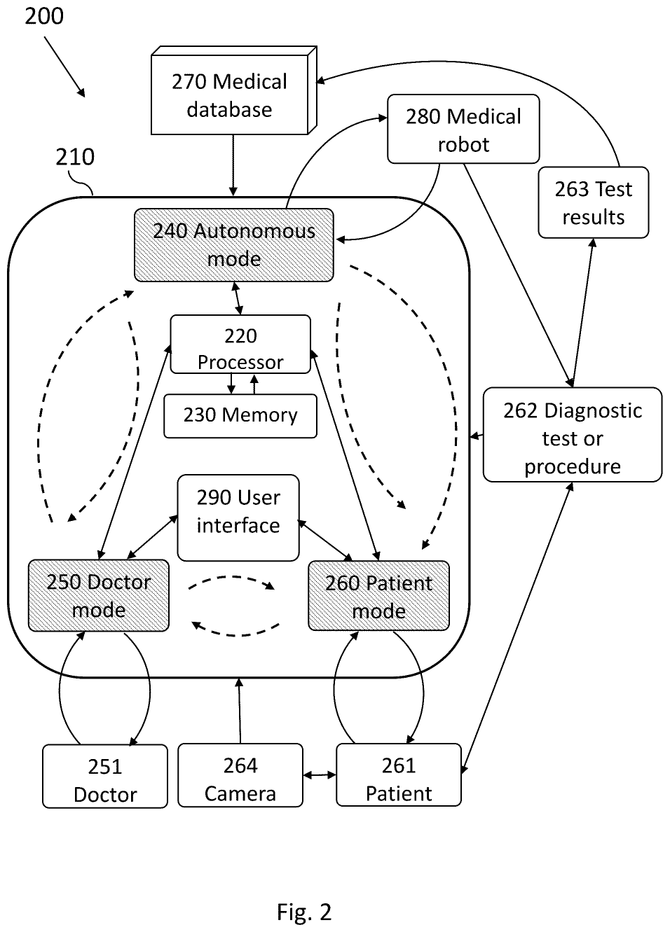

Reference is now made to FIG. 2, a schematic representation of an exemplary system for use as a comprehensive medical robotic system 200, showing the flow of information. The system comprises a controller 210, which includes a memory 230 and a processor 220, and three distinct modes of operation, 240, 250, and 260. The ‘modes of operation’ are drawn in the same form as the hardware components of the system, though it is to be understood that they are routines or algorithms which are implemented by the hardware components. Multiple integrated loops of information are exchanged, for example, between autonomous mode 240 and doctor decision-making mode 250; between autonomous mode 240 and patient decision-making mode 260; between doctor 251 decision-making mode 250 and patient decision-making mode 260. The controller 210 draws information from at least one medical database 270. Exchange of information also takes place between the system 200 operating in autonomous mode 240 and a medical robot 280, which may be instructed by the system to perform a diagnostic test or procedure 262 on the patient 261. The results 263 of the test or procedure are then reported back to the system 210 and the data from the test are added to the medical database. Doctors or other medical care providers 251 and patients 261 interact with the system via at least one user interface 290. Information obtained in any mode is made available to the other modes, and iterative operation of the entire system 200, or two of the three modes, improves the diagnostic accuracy of the operation. The camera 264 provides inputs to the system, and the camera may alternatively be comprised within a medical robot. Arrows contacting a specific box or element indicate electronic or physical input or flow of information between elements in the direction of the arrow, either unidirectional or bidirectional, according to the direction of the arrow. Arrows, shown as dashed lines, that connect two elements, albeit without direct contact of the arrow with the box, indicate alternate modes of operation.

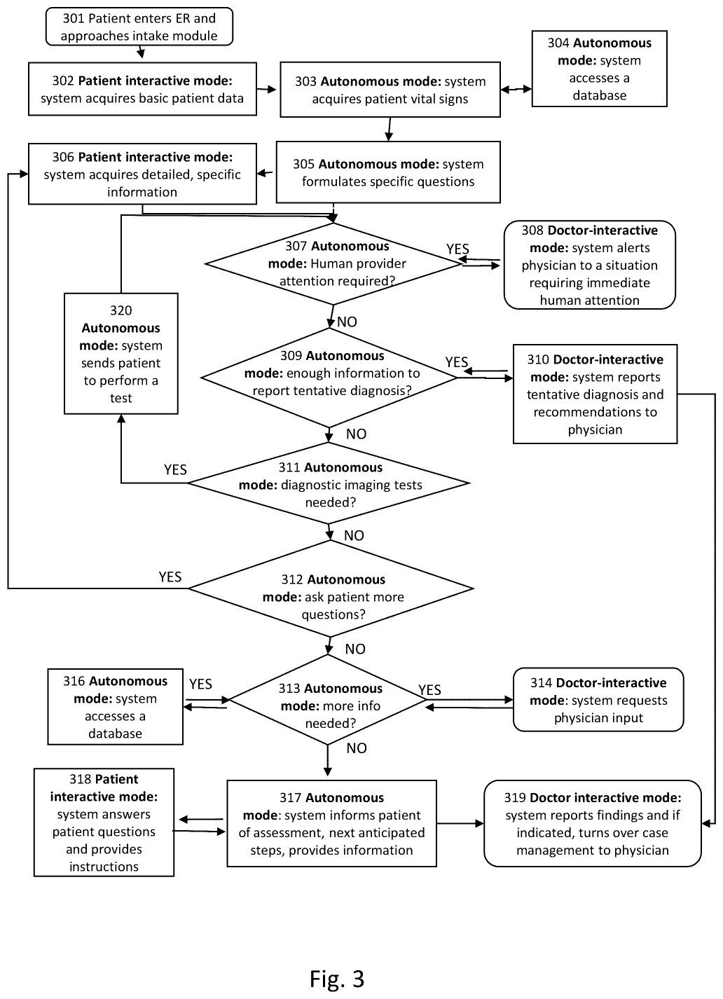

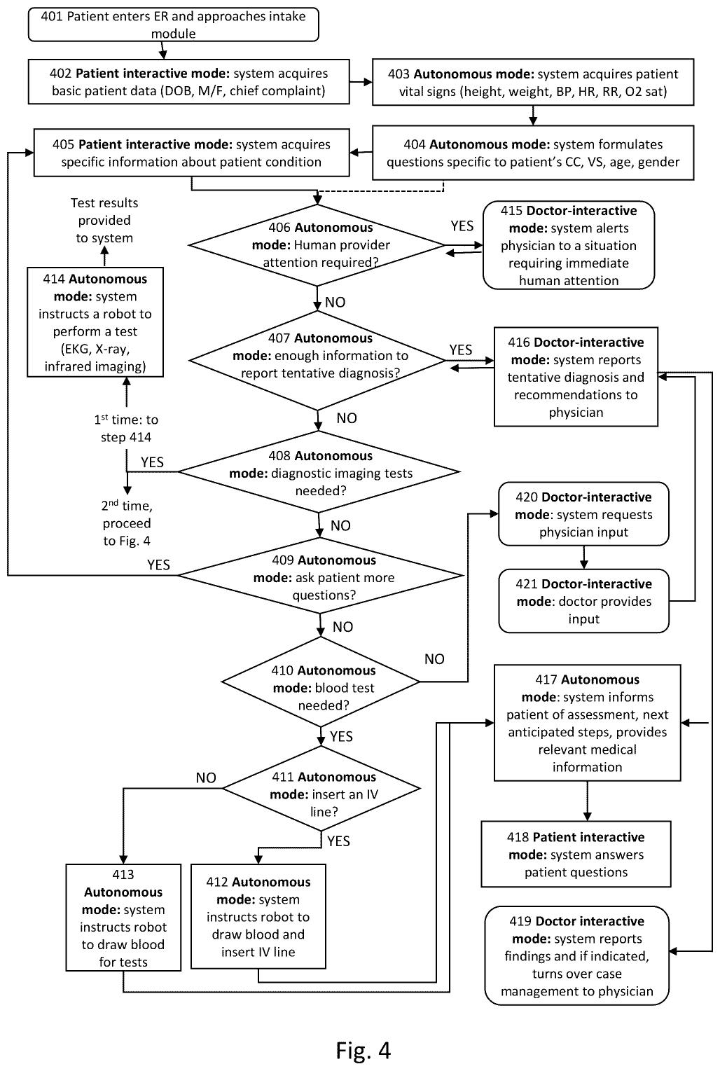

FIG. 3 shows an exemplary manner in which the system may use the three modes of operation to access and process information. More details of each individual step are shown in a slightly different format in the flowchart of FIG. 4 below. In step 301, the patient approaches the intake module. In step 302, the system operates in patient interactive mode, then switches in step 303 to autonomous mode to acquire data from the patient and, optionally in step 304, from a medical database. Such data may comprise, for example, past medical records of the patient under evaluation. In step 305, the system formulates specific questions based on the data acquired and analyzed in the previous steps. Based optionally and additionally on the patient responses in step 306, the system in step 307 decides whether to involve a health care provider. If so, bidirectional information exchange occurs with the physician in step 308. The data produced from this interaction is then analyzed, possibly in conjunction with the previously acquired raw and processed information. At each subsequent step of operation, the clinical picture becomes clearer as more information is exchanged, analyzed, and made available to the controller. In step 309, if enough information is available to make a diagnosis in autonomous mode, the system reports in step 310 to the health care provider. It is to be understood that providing a definite or tentative diagnosis could occur at many points along the illustrated path. In step 311, the system decides whether to order diagnostic tests, which leads to a loop with step 320 and returning to step 307; although the system could return to step 308 or to a different step using either autonomous or doctor-interactive modes. The system at any point, for example step 312, may decide to ask the patient additional questions, may decide in step 313 that more information is needed, and either query the database in step 316, or access doctor-interactive mode in step 314. In step 317, or in a subsequent step depending on how many iterations of mode operation are required and how much information needs to be obtained and analyzed, the system may provide a diagnosis and treatment plan, or provide recommendations to a physician in step 319. In some implementations, the system may handle the patient encounter without accessing doctor interactive mode. For example, in step 318, the system provides the patient with answers to questions, instructions, and may discharge the patient to home.

FIG. 4 is a flowchart showing an exemplary method used in the systems of the present disclosure, as shown in outline in FIG. 3 above, but now incorporating more details and decision processes than the outline of FIG. 3. The chart provides an overview of the manner in which the robotic control system would carry a patient through the emergency room experience, from initial entry to the point of autonomously finalizing a diagnosis, developing a treatment plan, providing patient instructions, or turning the patient over to a medical provider. It is to be understood that such a flowchart highlights only the main components or steps of the procedure, and only one possible implementation of the system. Such a complex and complicated decision-making process with input from artificial intelligence and other data sources may necessitate many more steps and decision points than can be illustrated in a figure. Also, depending on the patient's condition, in alternative embodiments of the present disclosure, some steps of the evaluation may be performed in a different order. It is also to be understood that in autonomous mode, any of the tests or measurements required may be performed, if circumstances prescribe, by medical staff.

In step 401 of the method, the patient enters the emergency department and approaches the patient intake system, a user interface for gathering initial patient information, which uses the control system operating in patient-interactive mode. In alternative implementations of the present invention, the medical setting may be a medical clinic. This interactive system is appropriate for those apparently reported 60% of emergency visits, in which the person requiring treatment is 1) ambulatory with or without assistance and capable of sitting or reclining in a chair or entry station, 2) conscious and able to respond to queries, or accompanied by a friend or relative knowledgeable of the patient's condition and able to interact with the control system and input accurate and reliable answers into the system, 3) in a stable physiological state, i.e., not at risk of rapid deterioration if not given immediate treatment. In alternative implementations of the present invention, the system, after evaluation of the patient, skips steps 401 and 402 and starts immediately with the acquisition of data such as vital signs in step 403.

In step 402, the system acquires basic data about the patient in any language in a patient interactive mode. Basic information regarding date of birth, gender, address, known drug allergies, chronic conditions, past history would all be acquired by entering the specifics or selecting from a list of choice. The data may, for example, be acquired in written format by touch-mediated selection or keyboard entry, or by voice recording. Alternatively, the data may be acquired by downloading an existing documentation of patient history from a cloud, server, mobile phone, computer, or the like, for example, upon identifying the patient via facial recognition. After the basic demographic and past history information is input or downloaded, the system may access records of any past admissions or emergency visits. This data may help in subsequent steps for decision-making in either autonomous or doctor interactive mode. Information regarding the patient's main complaint and symptoms may be entered according to a physiological system. The patient data may be stored in the memory of the controller for future use.

In step 403, when sufficient patient data has been collected, the system switches to the autonomous mode to acquire patient vital signs including such parameters as height and weight of the patient using a scale and measurement device that may be conveniently inbuilt into a robotic patient intake station, blood pressure and heart rate using an automatic cuff, respiratory rate and oxygen concentration using a pulse oximeter, and temperature using a non-contact thermometer. All of these devices may be used in cooperation with the system. Once sufficient vital signs data has been collected, the system operating in autonomous mode in step 404 will analyze the data acquired in steps 402 and 403, to formulate specific questions likely to lead to a diagnosis, treatment plan, or useful knowledge extraction based on the patient's main complaint, vital signs, age, gender, past history and on any other relevant information gleaned from a medical database and based on artificial intelligence.

In step 405, after the analysis is complete to the extent possible with the current collected data, the system switches to patient interactive mode to acquire specific information about the subject's condition based on the answers provided by the subject in step 402. It is understood that some patients are more articulate, more aware, and more capable of providing specific and accurate information about their condition and complaints than are others. Such a limitation may be overcome by making the data acquired in this step only one component of the total assessment and not highly dependent on the patient's mental capabilities or mental status and/or by using the nature of the inability or difficulty of the patient to respond as a factor for diagnosis. For this reason, the user interface should be as simple as possible, and the system should be aware of the quality and level of input provided by the patient or his/her representative. If the system determines that the input provided by the patient is inconsistent or unhelpful, it would be able to tailor the questions to a simpler level, ask the same question in different formats, or request input in pictorial format, e.g., show a pain scale from 1 to 10 with pictures to represent different levels of pain. Alternately or additionally, if the quality of the patient input is poor, system may ask the patient if there is someone assisting him/her that may be able to help answer the questions, or switch to doctor collaborative mode and request assistance from a health care provider.

Based on the patient's responses to initial symptom-based questions, the system continues asking more and more detailed and specific questions as needed. For example, if an otherwise healthy and active patient presented with a high spiking fever and other specific symptoms, the system might suspect exposure to an exotic infectious agent and could ask questions regarding recent activities including travel, identify endemic infectious conditions in the distant location, and present a likely differential diagnosis.

According to alternative methods of the present invention, if the system in step 104 assesses from the information in steps 402, 403, and/or 404 that the patient is in an unstable, rapidly deteriorating, or medically dangerous situation, the method bypasses step 405 and goes directly to step 406, as shown in the dashed path. Some examples of situations requiring such a response might include systolic blood pressure above a certain reading, fever greater than a predetermined cutoff temperature, heart rate above an age-specific and age-appropriate number, unresponsiveness, sudden change in mental status or appearance of deterioration. The deterioration may be determined either by a sudden change in monitored parameters, or by real-time images acquired via an associated camera, as shown in FIG. 2. The camera may be an optical device for acquiring images of the whole patient, or may be adapted to identify and image only the face; alternatively, the camera may use infrared, movement detection, blood flow velocity, or any other measureable change to identify a shift in the patient's status requiring immediate attention by a health care provider. The camera 264 feeds images to the system 210 for evaluation by the controller.

In step 406, the system operates in autonomous mode to decide if the patient requires a direct referral to a medical provider, based on physical signs as described above and alternatively or additionally, based on patient responses in step 405. Step 406 may additionally or alternatively comprise comparing the data and test results obtained from the patient to gathered data from multiple patients, and based on that information, performing a triage or prioritization for medical provider treatment of the patient relative to one or more other patients based on the severity of the patient's condition. This determination is an important part of the system design, because of the critical importance of triage and identifying patients in need of the most urgent care. The system may, for example, be able to distinguish between subjective input from a patient trying to bypass the wait by recording extreme responses, e.g., extreme pain levels, and patient input indicating a true medical emergency. The system may consider the limited number of human medical staff present in a particular medical setting.

If the system makes a decision in step 406 to alert the medical provider, in step 415 the system alerts the physician of a situation requiring at least some immediate human attention and turn care of the patient over to the medical provider and system in doctor interactive mode. “At least some immediate human attention” may be as minimal as comforting a patient who is upset, or may be as complex as preparing for emergency surgery. Before the medical provider and system take over care, the medical provider would have the option of asking questions and interacting with the system. Subsequently the medical provider may assess the patient together with the system and administer appropriate treatment. This is more advantageous than merely alerting a medical provider, since the doctor collaborative mode is more powerful and more likely to arrive at an accurate diagnosis than a doctor acting alone. If a determination were made by the doctor that the robotic control system would be helpful to continue the assessment, the physician may instruct the control system to continue the assessment at a specific point in the process determined by the doctor.

In step 406 of the method, if the system determines that no emergency alert is warranted and/or that autonomous mode is capable of continuing to assess the patient, the method continues to step 407, in which the system in autonomous mode makes an assessment of whether enough information had been collected and evaluated to make a tentative diagnosis. If so, the method proceeds to step 416 in doctor interactive mode. Alternatively, the system may store the tentative diagnosis for later use and remain in autonomous mode, since the medical provider may access outputs made when the system is in autonomous mode.

In step 416, the system reports a tentative diagnosis and/or make recommendations to the doctor for next steps in the assessment of the patient in doctor interactive mode based on at least some of 1) the input collected from steps 402-405, 2) information in an extracted form from a generic medical database, 3) past medical outcomes and patient records from the specific patient and others, for example, treated in the same department, by the same doctor, or in other area hospitals, and 4) artificial intelligence or machine learning and learning from past experience, such as using a database with data from previous patients with the same symptoms, or earlier ER visits by the same patient. Upon receiving the tentative diagnosis, the medical provider may ask further questions to the system regarding the patient's condition, request that the system obtain more information from a database, or direct the system to search for further diagnostic possibilities. In this step, not shown in FIG. 4 but understood to comprise further operation of the system continuing from step 416 or any other step using doctor-interactive mode, the robotic control system and the medical provider are having a “conversation” regarding the best course of action for the patient under evaluation, which may include verbal dialogue, outputs on a display, keyboard or touchscreen input, and the like. The system may recommend a CT scan, invasive radiological test, e.g., angiography, or other evaluation. The system may also recommend hospitalization, release with or without a course of treatment, or other action. At a point mutually determined by the doctor and the control system or by the doctor alone, the doctor takes over sole care of the patient, in step 419. This may occur when the doctor and system have mutually arrived at a diagnosis that the doctor is satisfied with.

In step 417, following drawing of blood with or without IV line insertion in step 412 or 413, the system communicates with the medical provider and optionally turns care of the patient over to one or more humans. This could occur in any of steps 415, 416, 419-421, or others. The system is not constrained by what is illustrated in FIG. 4, which exemplifies a limited representation of one possible implementation. It is difficult to diagram all of the steps in system operation in a single diagram. In some alternative embodiments of the present invention, the system may return to autonomous mode, or patient collaborative mode, to inform the patient of its assessment and next anticipated steps in the treatment protocol. The system further provides relevant medical information regarding the patient's condition to the degree Known and appropriate for a given situation. In step 418, the system enters patient interactive mode and answers questions the patient might have regarding his/her condition, the probable course of treatment, or other aspects of the visit. The questions may be provided by the system or may be entered by the patient, using word-recognition software and commonly employed search algorithms, tailored and possibly limited by the information acquired and collected by the system regarding this specific case.

In step 407, operating in autonomous mode, if the system determines that it lacks sufficient data to make a tentative diagnosis and that further tests are warranted which it is capable of performing in autonomous mode, the system will proceed to step 408 in autonomous mode. In step 408, continuing in autonomous mode, the system will decide whether to instruct a medical robot to perform one or more diagnostic medical tests such as ECG; x-ray of a specific part of the body, e.g. to assess a potential fracture; or infrared imaging to evaluate temperature, blood flow to a specific region, or other diagnostic purpose. If the decision is made to instruct a robot to perform a test, the system proceeds to step 414 in autonomous mode.

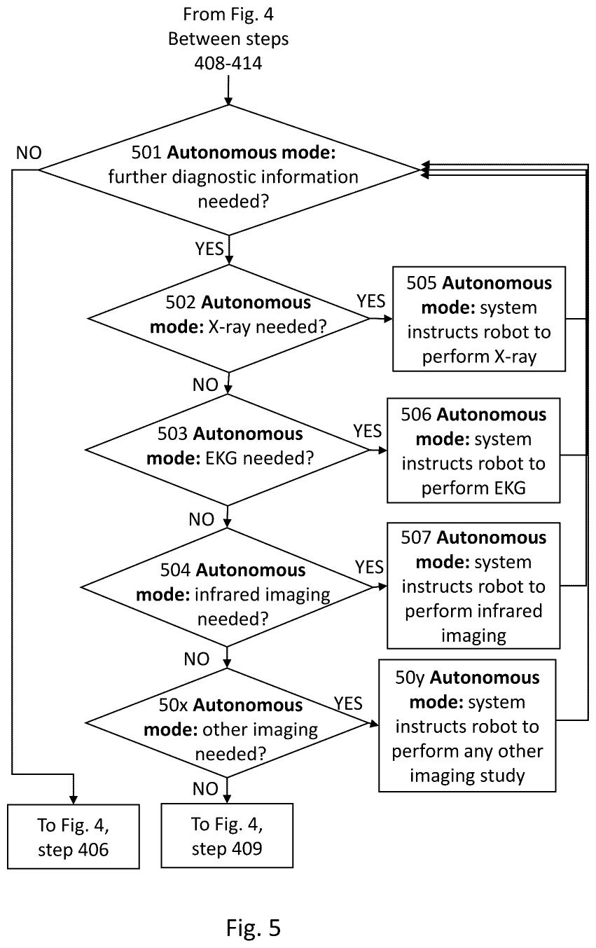

In step 414, at least one diagnostic test is performed. While the system operates in autonomous mode, the system simultaneously provides instructions as needed to the patient regarding positioning of him/herself or body parts to undergo the test, e.g., placing a limb to be x-rayed in a specific pose. The tests listed in step 408 are all exemplary tests that could be built into a robotic control system intake module, such that the patient under evaluation may not need to move from his/her position during the entire intake evaluation, which may be from initial entry into the ER until the system outputs a diagnosis. Such a robotic system intake module may comprise any of a camera, a display, and a keyboard, a touchscreen, a haptic interface, a voice recognition system with a microphone, or other means for the patient to input information or questions. Further details of the specific tests and their order of performance are detailed in FIG. 5.

The results of the test or tests performed in step 414 are evaluated by the system in autonomous mode to identify pathological features requiring medical attention. If the same patient had previously been treated in the same department or hospital, or if electronic medical records were available from another source, the system may compare the current images or results with the previous set to identify changes potentially indicative of a progressive or new pathological process. The system may use machine learning or artificial intelligence to identify normal and abnormal features of an imaging study or dynamic evaluation, and use this information to formulate the next step of the evaluation. At this point, the system returns to step 406 in autonomous mode, adds the information obtained in step 414 or from the steps described in FIG. 5, and assesses whether referral to a human medical provider may be warranted.

Returning to step 406 and operating in autonomous mode, the system proceeds iteratively as described above until in step 408 no further diagnostic tests are indicated that the system could perform in autonomous mode. In this eventuality, the system continues in autonomous mode and the method proceeds to step 409, where the system makes a decision as to whether more patient input is required. If positive, the system returns to step 405 in patient interactive mode.

Although not shown in FIG. 4, the method according to alternative embodiments of the present invention returns to one or more prior steps at any point in the evaluation. For example, after step 414, the method may, based on a system decision, go back to step 405 to operate in patient interactive mode and acquire more specific and relevant information from the patient based on the results of the imaging studies or ECG. If, e.g., the patient presented with shortness of breath and rapid respiration, the system may decide to acquire a set of chest x-rays to evaluate for pneumonia. If no infiltrate or opacity were observed but a broken rib was identified, the system may decide to direct the investigation in a different direction to identify the cause of the fracture and possible associated injuries.

If in step 409, the system decides that no further patient input is warranted, the system proceeds in autonomous mode to step 410 of the evaluation and assess whether blood tests, or collection of other body fluids, are required. It is understood that, based on the results of earlier steps, the system may decide to order blood tests, step before performing imaging tests in step 414. Blood tests that could be ordered are any of the commonly ordered sets to determine levels, distribution and types and other parameters of red and white blood cells; sodium and other ion levels (chemistry panel); levels of alcohol, other drugs or suspected toxins, blood cultures, or other diagnostic blood test. The decision on the best order of performing tests would be made based on the most critical information needed, and on other practical considerations, e.g., mobility of the patient's limbs before and after possible insertion of an intravenous line, and the length of time required to return results of the blood test or imaging study.

In step 410, if blood tests are not indicated, the system proceeds to step 420 and enters doctor interactive mode. In step 420, the system may make suggestions, present evaluation results until this point, ask questions, or request further input from the physician as to additional diagnostic tests to be performed. The physician may review the data and decide to take over care of the patient, or ask the system to search for other information such as medical case reports or articles on PubMed that either physician or robotic control system could then use to contribute to decisions, or which both could use to collaborate on a desired treatment plan. The physician may also provide input that would allow the system to perform additional evaluations in autonomous mode. Although not shown in the figure, the system could proceed from step 120 to step 119 at this point and turn case management over to the physician.

If blood tests are indicated based on the autonomous mode evaluation in step 410, the system proceeds to step 411 and determines whether to insert an intravenous (IV) line at the time of blood drawing. The decision to insert an IV line may depend on factors such as the likelihood that the patient would require IV fluids, IV medicines, or other indication for venous access. The decision to insert an IV line may be based on the probability of the anticipated need for venous access, and on the ease of inserting an IV line. The ease of insertion may depend on factors such as the age of the patient, patient cooperation, hydration status. If the system, for example, decides the likelihood of requiring venous access warrants insertion of an IV line and simultaneously determines that the likelihood of success in line insertion is greater than a defined probability, the system will proceed to step 412.

In step 412, the system continues to operate in autonomous mode to instruct a medical robot to draw blood and insert an IV line of the appropriate gauge. This task may be accomplished, e.g., by a robotic arm, in some embodiments attached to the robotic control system, the robotic arm having infrared capability to detect the vein and ultrasound imaging to detect blood flow. Any such system could be conveniently adapted for this purpose, such as that manufactured by Veebot. If in step 411, the system determines not to insert an IV line, the method will proceed to step 413 and the system instructs the medical robot to draw blood as described in step 412, omitting IV insertion. In the course of these steps, 412 and 413, the control system operates autonomously, but preferably provides feedback to the patient as to the next steps of the process and give instructions as needed, for example, to position the patient's arm in a suitable location for the phlebotomy. These instructions may be either auditory, visual, tactile, or a combination of all three.

At this point of the evaluation, the system determines whether to return to any previous step, and whether to provide the patient with information in step 417, answer any questions in step 418, wait for the results of the blood tests, or proceed to step 419 in doctor interactive mode and report the current findings and results to the medical provider, for example, highlighting any abnormal test results, providing a differential diagnosis and suggesting a course of treatment or further evaluation. Here the control system and the doctor interact as in step 420 described above, until a mutual decision is made to turn care of the patient over to the human provider, which may occur, for example, when the doctor and system mutually arrive at the same diagnosis.

FIG. 5 illustrates a more detailed implementation of step 414 of FIG. 4, in which the system autonomously performs one or more tests such as an ECG, x-ray, infrared imaging, or other study that may be performed without transferring the patient and without medical staff intervention.

In step 501, the system decides if further diagnostic information is indicated. If yes, the system may then decide for instance which imaging test would be most informative for arriving at a tentative or conclusive diagnosis, for example, by going through the list of limited available options. This decision is based on the information acquired in previous steps, and whether and at what point a clear diagnostic picture begins to emerge. For example, if the patient presents with a fever, high blood pressure, and shortness of breath, if the fever is very high, above a given temperature, and the blood pressure is only mildly elevated, the system may decide first to perform an x-ray to rule out pneumonia. However, if the fever is mild and the blood pressure highly elevated, the system may decide first to perform an ECG. This example is merely for the purposes of explaining how the system would operate, and is not meant to limit the scope of the system's capabilities.

In step 502, the system decides if an x-ray is needed, and if so, proceeds to step 505 where the system instructs a medical robot to perform the x-ray. Based on an autonomous evaluation of the imaging results in autonomous mode, the method returns to step 501 where the system determines if further diagnostic studies are indicated. If not, the method returns to step 409 of FIG. 4. If yes, the method proceeds to step 203 to evaluate whether to perform an ECG. If yes, the system instructs a robot to perform the EKG in step 506; if not, the method proceeds to step 504 to evaluate the need for infrared imaging. If yes, the imaging would be performed in step 507; if not, the system would proceed to further iterations of decisions regarding other medical tests in step 50x, and in performance of them in step 50y. This process could continue by the system in autonomous mode until a clear result was obtained or the system ran out of potential diagnostic tests that it could instruct a robot to perform. Again, based on the individual patient's clinical picture, the system would prioritize tests according to those most likely to provide diagnostic information and the importance of a given test result for a successful clinical outcome.

As stated above, the flow of information in FIG. 4 is meant to be an exemplary implementation of a method according to the invention, and variations on this specific scheme could be adapted, based on a given patient's needs, on system learning over time, and on advances in artificial intelligence. The system may be programmed to operate in a curtailed mode, for example, if an emergency situation such as a natural disaster or terror attack placed intense demands on the resources of a given emergency department, such that many patients needed rapid assessment and triage at once. The system may be programmed to learn, based on number of patients waiting, how much time to allocate to an individual. The system may make tentative diagnoses based on limited information in times of great demand. In other circumstances, the system may perform an extensive analysis and evaluation and provide a detailed assessment and list of differential diagnoses, with weights given to each possible diagnosis. When a given patient has a complex condition and has been evaluated thoroughly by physicians, the system may be asked to provide a detailed assessment that could take considerable time to perform, with extensive mining of electronic data sources and literature searches. For example, the system may utilize a higher representation of the autonomous mode versus the doctor in making decisions, and/or make autonomous mode a higher priority than doctor collaborative mode, with suggested time limitations for doctor collaborative mode based on severity of one or more patient conditions.

The system may evaluate multiple patients simultaneously in a specific mode of operation. In a most efficient operation, the system may take a patient from his/her initial presentation to the emergency room in step 101 to a tentative or conclusive diagnosis in a matter of under an hour. It is even possible that, for a clear-cut case, the system may be able to suggest a course of outpatient treatment, write a prescription, and send the patient homeward with minimal if any input from a medical provider. In such a scenario, at the time of discharge, the system may make notes in the patient's electronic medical record by storing information in the memory to the effect that if this patient returned to the emergency department within a specified time period, a specific course of action should be immediately taken. Patients sent home may be provided by the system operating in autonomous mode with a list of instructions for self-care, routine follow up, and signs indicating a need for further immediate return to the emergency department for medical care.

It is appreciated by persons skilled in the art that the present invention is not limited by what has been particularly shown and described hereinabove. Rather the scope of the present invention includes both combinations and subcombinations of various features described hereinabove as well as variations and modifications thereto which would occur to a person of skill in the art upon reading the above description and which are not in the prior art.

I claim:

1. A system for use in a medical care environment, the system comprising:
a controller, comprising:
(a) a patient collaborative decision-making mode in which the system exchanges data with a patient;
(b) an autonomous decision-making mode in which the system is fully autonomous; and
(c) a doctor collaborative decision-making mode in which the system exchanges data with at least one health care provider;
the controller being configured to alternate among the modes to:
(i) analyze, using artificial intelligence, data obtained from at least one of: the patient, the at least one health care provider, at least one clinical procedure, at least one diagnostic test, or at least one medical database;
(ii) use the analyzed data to select at least one diagnostic test, clinical procedure, or clinical recommendation to be performed by either a health care provider or a medical robot; and
(iii) if indicated by an output from the analyzed data, autonomously instruct the at least one medical robot to perform at least one of a clinical procedure or a diagnostic test;
wherein iterative operation of the system, comprising at least one alternation between at least two of the modes, achieves a diagnosis of the patient, and at least one of a treatment plan or instructions to the patient.

2. The system according to claim 1, wherein the controller is further configured to do at least one of:
(i) in the autonomous decision-making mode, improve decision-making capabilities of the robotic control system based on at least one of:
a) the data,
b) statistical analysis performed by the robotic control system, and
c) the diagnostic tests performed by the at least one medical robot;
(ii) in the doctor collaborative mode, incorporate the expertise of the at least one health care provider with the data and with the statistical analysis of the robotic control system to improve collaborative decision-making capabilities; and
(iii) in the patient collaborative mode, output questions to the patient likely to lead an accurate diagnosis,
wherein the learning paths for autonomous mode, doctor collaborative mode, and patient collaborative mode are different.

3. The system according to either of claim 1 or 2, wherein at least one of collected patient data, analyzed information, doctor judgments/decisions, and system judgments/decisions, is available to doctor collaborative mode, patient collaborative mode, and autonomous mode for use in at least one of i) making further judgments/decisions and ii) as training set data for artificial intelligence algorithms.

4. The system according to either of claim 2 or 3, wherein the system is configured to arrive at the same diagnosis for the patient via a first method when the system is in a first mode or first sequential combination of modes, and via a second method when the system is in a second mode or second sequential combination of modes.

5. The system according to any of the previous claims, wherein the robotic control system is configured to provide information to the patient in response to a patient query in at least one of the patient interactive mode and the autonomous mode.

6. The system according to any of the previous claims, wherein the robotic control system in autonomous mode is adapted to exchange analyzed data with, and to learn from, any number of other control systems.

7. The system according to any of the previous claims, wherein the controller is configured to be used in at least one of the modes for long-term patient monitoring in a health care setting.

8. The system according to any of the previous claims, wherein the controller is configured in the autonomous mode to instruct the at least one medical robot to perform any number of diagnostic procedures, comprising at least one of blood drawing, imaging studies, and vital sign acquisition.

9. The system according to any of the previous claims, wherein the controller in the autonomous mode is configured to instruct at least one of the medical robots to perform any number of therapeutic procedures, comprising at least one of prescribing or dispensing medication; providing written or oral instructions; and administering IV fluids.

10. The system according to any of the previous claims, wherein, if iterative sharing of the exchanged, analyzed data between the patient collaborative mode and the autonomous mode results in different output from iterative sharing of the exchanged, analyzed data between the doctor collaborative mode and the autonomous mode, comparison of output from the modes enables achievement of improved diagnostic accuracy.

11. The system according to any of the previous claims, wherein iterative operation of the controller uses artificial intelligence to reach at least one of the diagnosis and the treatment plan, and the patient instructions.

12. The system according to any of the previous claims, wherein the artificial intelligence comprises machine learning.

13. The system according to any of the previous claims, wherein the controller, based on the iterative operation, is configured to switch among the modes, and wherein the decision of at least one of whether to switch to a different mode, when to switch to a different mode, and which mode to switch to, is made by the system using at least collected patient data and artificial intelligence, such that diagnostic accuracy is improved.

14. The system according to any of the previous claims, wherein the instructions to the patient comprise any of: to fill a prescription, to take a prescription, to return for a scheduled follow-up visit, to carry out routine home care tasks, to contact a health care provider, to make an appointment, to allow a medical robot to carry out a test such as blood drawing, to transfer to a different department, or other instructions that are routinely provided to a patient by a human provider in a medical setting.

15. The system according to any of the previous claims, further comprising at least one user interface adapted to exchange the data between the controller and at least one of the patient and the health care provider.

16. The system according to any of the previous claims, wherein said data comprises any of the patient's current age, gender, height, weight, BMI, blood pressure, heart rate, blood laboratory test results, imaging study results, biopsy test results, a previous medical diagnosis, or the patient's past medical data.

17. The system according to any of the previous claims, wherein the analyzing of the data comprises at least one of computational statistics, data mining using exploratory data analysis, or data mining through unsupervised or supervised learning, the data being obtained from at least one of the patient, the health care provider, the patient's previous medical records, the at least one medical database, or previous learning by the robotic control system.

18. The system according to any of the previous claims, wherein the alternation among the modes is determined by the system obtaining maximum data/information from a given mode, whereupon it switches to a different mode, the controller beginning its operation in the patient collaborative mode, switching to the autonomous mode, and, if indicated, switching to the doctor collaborative mode.

19. A method for automated assessment of a patient, comprising the steps of:
i) providing a controller capable of iteratively alternating among three decision-making modes, the modes comprising: a patient information-exchange mode, an autonomous mode, and a health care provider information-exchange mode,
ii) using the controller to obtain data from the patient and from one or more medical databases,
iii) using artificial intelligence and statistical analysis capabilities of the controller, analyzing the data, at least one of the data or the analyzed data being exchanged among at least two of the modes;
iv) using iterative operation of the controller to:
a) in the autonomous decision-making mode, make decisions based on i) the analyzed information and ii) input from at least one of the collaborative modes;
b) in the health care provider information-exchange mode, make decisions based on the analyzed information and incorporating the expertise of at least one health care provider; and
c) in the patient information-exchange mode, output questions, analyze answers provided by the patient, and provide instructions to the patient;
v) performing at least one iterative operation in at least two of the modes; and
vi) arriving at a diagnosis of the patient, and at least one of a treatment plan or patient instructions.

20. The method according to claim 19, wherein the controller is further configured to do at least one of:
(i) in the autonomous decision-making mode, improve decision-making capabilities of the robotic control system based on at least one of:
a) the data,
b) statistical analysis performed by the robotic control system, and
c) the diagnostic tests performed by the at least one medical robot;
(ii) in the doctor collaborative mode, incorporate the expertise of the at least one health care provider with the data and with the statistical analysis of the robotic control system to improve collaborative decision-making capabilities; and
(iii) in the patient collaborative mode, output questions to the patient likely to lead to an accurate diagnosis,
wherein the learning paths for autonomous mode, doctor collaborative mode, and patient collaborative mode are different.

21. The method according to either of claim 19 or 20, further comprising the step of providing at least one of the collected patient data, analyzed information, and controller judgments/decisions, to doctor collaborative mode, patient collaborative mode, and autonomous mode for use in at least one of i) making further judgments/decisions and ii) as training set data for artificial intelligence algorithms.

22. A system according to any of claims 19-21, wherein due to different learning paths, the method of arriving at a diagnosis in a first mode or first sequential combination of modes, are different for a second patient with the same symptoms in the same situation.

23. The method according to any of claims 19-22, further comprising the step of providing information to the patient in response to a patient query in at least one of the patient interactive mode and the autonomous mode.

24. The method according to any of claims 19-23, further comprising the step of the controller in autonomous mode exchanging analyzed data with, and learning from, any number of other controllers.

25. The method according to any of claims 19-24, further comprising the step of the controller in autonomous mode instructing at least one medical robot to perform any number of diagnostic procedures, comprising at least one of blood drawing, imaging studies, and vital sign acquisition.

26. The method according to any of claims 19-25, wherein, if iterative exchange of the exchanged, analyzed data between the patient collaborative mode and the autonomous mode results in different output from iterative exchange of the exchanged, analyzed data between the doctor collaborative mode and the autonomous mode, comparison of output from the modes achieves improved diagnostic accuracy.

27. The method according to any of claims 19-26, wherein iterative operation of the controller uses artificial intelligence to reach the diagnosis and the treatment plan, or the patient instructions.

28. The method according to any of claims 19-27, wherein the artificial intelligence comprises machine learning.

29. The method according to any of claims 19-28, wherein the controller, based on the iterative operation, switches among the modes, and wherein the decision of at least one of i) whether to switch to a different mode, ii) when to switch to a different mode, and iii) which mode to switch to, is made by the controller using at least collected patient data and artificial intelligence, such that diagnostic accuracy is improved.

30. The method according to any of claims 19-29, wherein the data comprises any of the patient's current age, gender, height, weight, BMI, blood pressure, heart rate, blood laboratory test results, imaging study results, biopsy test results, a previous medical diagnosis, or the patient's past medical data.

31. The method according to any of claims 19-30, wherein the analyzing of the data comprises at least one of computational statistics, data mining using exploratory data analysis, or data mining through unsupervised learning, the data being obtained from at least one of the patient, the health care provider, the patient's previous medical records, the at least one medical database, or previous learning by the controller.

32. The method according to any of claims 19-31, wherein the alternating among the modes is determined by the controller obtaining maximum data/information from a given mode, whereupon it switches to a different mode, the controller beginning its operation in the patient collaborative mode, switching to the autonomous mode, and, if indicated, switching to the doctor collaborative mode.

33. The system according to claim 19, wherein the diagnosis comprises any one of a provisional diagnosis, a differential diagnosis, or a final diagnosis.

34. The system according to claim 1, wherein for a given patient with given symptoms, a first mode or first sequential combination of modes, and a second mode or a second sequential combination of modes, are configured to perform different methods that are driven by different optimization goals or optimization strategies yet arrive at the same ultimate treatment plan or diagnosis.

35. The system according to claim 33, wherein the first mode or sequential combination of modes improves diagnosis over time using artificial intelligence according to a first optimization goal or strategy, and wherein the second mode or sequential combination of modes improves methods over time using artificial intelligence according to a second optimization goal or strategy, such that diagnostic accuracy is improved.

36. The system according to any of claims 1-18, wherein the diagnosis comprises any one of a provisional diagnosis, a differential diagnosis, or a final diagnosis.
